(19) United States
(12) Patent Application Publication
(10) Pub. No.: US 2007/0196235 A1
(43) Pub. Date: Aug. 23, 2007

(54) Ultraviolet radiation-based media purification

(76) Inventors: Michael Shur, Latham, NY (US); Remigijus Gaska, Columbia, SC (US); Yuriy Bilenko, Columbia, SC (US)

Correspondence Address: HOFFMAN WARNICK & D'ALESSANDRO, LLC, 75 STATE STREET, 14TH FLOOR, ALBANY, NY, 12207, US

(21) Appl. No.: 11/180,495

(22) Filed: Jul. 13, 2005

Related U.S. Application Data
(60) Provisional application No. 60/588,153, filed on Jul. 15, 2004.

(51) Int. Cl. A61L 9/18; A61L 9/20

(52) U.S. Cl. 422/62; 422/121; 250/455.11

(57) ABSTRACT

An improved solution for purifying a medium using ultraviolet radiation is provided. The ultraviolet radiation is generated using at least one of an ultraviolet light emitting diode or an ultraviolet laser diode. In one embodiment, the diode(s) are disposed on a conduit that contains the medium and through which the medium moves. In this manner, operation of the system is made safer and the system can be incorporated into various new purification applications.

### CROSS-REFERENCE TO RELATED APPLICATION

The current application claims the benefit of co-pending U.S. Provisional Application No. 60/588,153, filed on Jul. 15, 2004, which is hereby incorporated herein by reference.

### BACKGROUND OF THE INVENTION

1. Technical Field

The invention relates generally to purifying a medium, and more particularly, to a solution for purifying a medium using ultraviolet radiation generated by an ultraviolet light emitting diode and/or an ultraviolet laser diode.

2. Background Art

Ultraviolet radiation has been successfully used in the purification (e.g., sterilization) of various media, such as air, water, and food. In general, it is desirable that the ultraviolet radiation comprises wavelength(s) that are close to the absorption peak(s) of biologically significant molecules of DNA and/or proteins of a target impurity. For example, impurities, such as a bacterium, a virus, a protozoan, a germ, etc., comprise DNA/proteins having corresponding absorption peaks. By exposing the DNA/proteins to ultraviolet radiation having a wavelength close to the absorption peak(s) for a sufficient time and at a sufficient power, the impurity is destroyed. To this extent, exposing a medium that includes one or more of these impurities to sufficient ultraviolet radiation can destroy some or all of the impurities. When sufficient impurities are destroyed, the medium is purified to a safe condition.

Typically, the source of the ultraviolet radiation in a purification system is a mercury lamp. To this extent, a low-pressure or a medium-pressure mercury lamp provides a linear spectrum of radiation with one or more peak lines having a wavelength that is in the relative vicinity to the DNA absorption line. For example, a low-pressure mercury lamp having a main peak at 253.4 nanometers (nm) is generally used in low-consumption residential water purification systems and residential air purification systems. Further, a medium-pressure mercury lamp having a higher radiation power and a multi-peak radiation spectrum are used in municipal systems with medium and high water consumption.

However, the use of a mercury lamp as the source of ultraviolet radiation has significant drawbacks. For example, mercury is an extremely dangerous element, thereby limiting the applications of mercury-based water purification systems. In particular, such a mercury-based water purification system is generally not used in transportation or individual applications. Further, a typical lifetime of the mercury lamp generally does not exceed ten thousand hours. Still further, the radiation spectrum of the ultraviolet radiation generated by the mercury lamp includes peak lines having characteristic wavelengths that do not exactly coincide with the absorption peaks of DNA and proteins. Additionally, these peak lines cannot be controlled or adjusted, resulting in a decrease in the efficiency of the purification. Still further, mercury lamps are fragile and bulky, which generally adds to the overall cost and/or size of the system. Various other limitations are present as will be recognized by one of ordinary skill in the art.

Some solutions seek to overcome one or more of these limitations. For example, a handheld ultraviolet water purification system that uses a miniature mercury lamp has been proposed. However, in this solution, the mercury lamp must contact and be manually steered in the water. The fragile nature of the quartz sleeve that includes the mercury lamp makes such a device dangerous for residential applications and not appropriate for transport, field, and portable applications.

In light of the above, a need exists for ultraviolet radiation-based media purification that overcomes one or more of the limitations of the prior art.

### SUMMARY OF THE INVENTION

The invention provides an improved solution for purifying a medium, e.g., a liquid (e.g., water, a biological fluid, or the like), a gas (e.g., air), or a solid (e.g., a food item, an object, or the like). In particular, the invention provides a system for purifying the medium in which ultraviolet radiation is generated using at least one of an ultraviolet light emitting diode or an ultraviolet laser diode. In this manner, operation of the system is made safer and the system can be incorporated into various new purification applications. In one embodiment, the diode(s) are disposed on a conduit that contains the medium and through which the medium moves. The system can further include a power module that is capable of adjusting one or more characteristics of the ultraviolet radiation and a control system that controls the ultraviolet radiation generation. The system can also include an evaluation system and/or a feedback system that can provide data on the medium, which can be used to adjust one or more characteristics of the ultraviolet radiation. One or more additional ultraviolet radiation source(s), such as a mercury lamp, and/or one or more non-ultraviolet radiation source(s), such as an X-ray radiation source, also can be included.

A first aspect of the invention provides a system for purifying a medium, the system comprising: a radiation module that includes a set of ultraviolet radiation sources for generating ultraviolet radiation to purify the medium, wherein the set of ultraviolet radiation sources comprises at least one of an ultraviolet light emitting diode or an ultraviolet laser diode.

A second aspect of the invention provides a system for purifying a medium, the system comprising: a conduit for containing the medium and allowing the medium to pass there through; and a set of ultraviolet radiation sources disposed on the conduit, wherein the set of ultraviolet radiation sources direct the ultraviolet radiation on the medium in the conduit.

A third aspect of the invention provides a system for purifying a medium, the system comprising: a radiation module that includes a set of ultraviolet radiation sources for generating ultraviolet radiation to purify the medium, wherein the set of ultraviolet radiation sources comprises at least one of an ultraviolet light emitting diode or an ultraviolet laser diode; a control system for operating the radiation module; and a transportation system for moving the medium through the radiation module.

A fourth aspect of the invention provides a method of purifying a medium, the method comprising: generating ultraviolet radiation using at least one of an ultraviolet light emitting diode or an ultraviolet laser diode.

A fifth aspect of the invention provides a method of purifying a medium, the method comprising: containing the medium in a conduit and allowing the medium to pass there through; and directing ultraviolet radiation on the medium using a set of ultraviolet radiation sources disposed on the conduit.

A sixth aspect of the invention provides a method of purifying a medium, the method comprising: generating ultraviolet radiation to purify the medium using a radiation module that comprises at least one of an ultraviolet light emitting diode or an ultraviolet laser diode; adjusting the at least one of an ultraviolet light emitting diode or an ultraviolet laser diode based on a property of the medium; and moving the medium through the radiation module.

The illustrative aspects of the present invention are designed to solve the problems herein described and other problems not discussed, which are discoverable by a skilled artisan.

### BRIEF DESCRIPTION OF THE DRAWINGS

These and other features of this invention will be more readily understood from the following detailed description of the various aspects of the invention taken in conjunction with the accompanying drawings that depict various embodiments of the invention, in which:

FIG. 1 shows an illustrative environment for purifying a medium;

FIG. 2 shows a more detailed view of an illustrative purification system; and

FIG. 3 shows an illustrative conduit comprising ultraviolet diodes disposed thereon.

It is noted that the drawings of the invention are not to scale. The drawings are intended to depict only typical aspects of the invention, and therefore should not be considered as limiting the scope of the invention. In the drawings, like numbering represents like elements between the drawings.

### DETAILED DESCRIPTION

As indicated above, the invention provides an improved solution for purifying a medium, e.g., a liquid (e.g., water, a biological fluid, or the like), a gas (e.g., air), or a solid (e.g., a food item, an object, or the like). In particular, the invention provides a system for purifying the medium in which ultraviolet radiation is generated using at least one of an ultraviolet light emitting diode or an ultraviolet laser diode. In this manner, operation of the system is made safer and the system can be incorporated into various new purification applications. In one embodiment, the diode(s) are disposed on a conduit that contains the medium and through which the medium moves. The system can further include a power module that is capable of adjusting one or more characteristics of the ultraviolet radiation and a control system that controls the ultraviolet radiation generation. The system can also include an evaluation system and/or a feedback system that can provide data on the medium, which can be used to adjust one or more characteristics of the ultraviolet radiation. One or more additional ultraviolet radiation source(s), such as a mercury lamp, and/or one or more non-ultraviolet radiation source(s), such as an X-ray radiation source, also can be included.

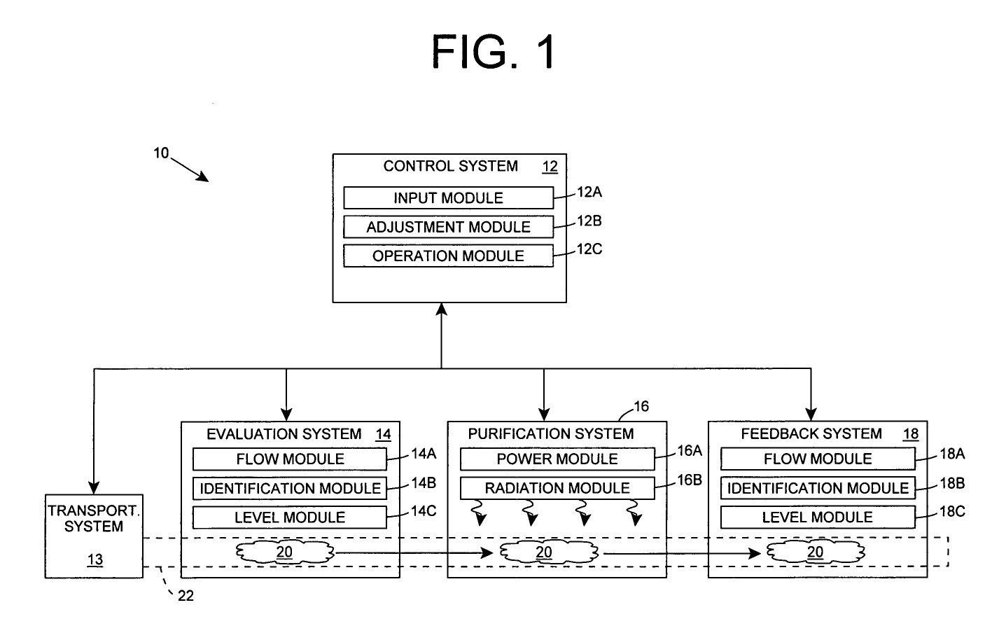

Turning to the drawings, FIG. 1 shows an illustrative environment 10 for purifying a medium 20. To this extent, environment 10 includes a control system 12 that obtains data from and/or controls a transportation (transport.) system 13, an evaluation system 14, a purification system 16 and a feedback system 18. Operation of each of these systems is discussed below. However, it is understood that environment 10 can be implemented without one or more of these systems or could include one or more additional systems. For example, environment 10 could lack control system 12, transportation system 13, evaluation system 14 and/or feedback system 18. Further, control system 12 and purification system 16 could be implemented as a single system, such as a handheld device. In any event, environment 10 is shown having the separate systems for clarity.

As noted above, control system 12 can control transportation system 13, evaluation system 14, purification system 16, and/or feedback system 18. To this extent, it is understood that control system 12 includes one or more input/output (I/O) devices for communicating with the various systems 13, 14, 16, 18. Further, control system 12 can include I/O device(s) for communicating with a user and/or one or more additional systems not shown. Communications between the various systems can occur over any combination of one or more types of wired and/or wireless communications links, such as a public or private network. Regardless, control system 12 can comprise any computing article of manufacture capable of performing the various process steps described herein. For example, control system 12 can comprise a general purpose computing article of manufacture capable of executing computer program code installed by a user (e.g., a personal computer, handheld device, etc.). Alternatively, control system 12 can comprise a specific purpose computing article of manufacture comprising hardware and/or computer program code for performing specific functions, a computing article of manufacture that comprises a combination of specific purpose and general purpose hardware/software, or the like. In each case, the program code and hardware can be created using standard programming and engineering techniques, respectively.

In any event, when control system 12 includes computer program code, it is understood that various components, such as a memory and processor, are included to enable the execution thereof. As used herein, it is understood that the terms “program code” and “computer program code” are synonymous and mean any expression, in any language, code or notation, of a set of instructions intended to cause a computing device having an information processing capability to perform a particular function either directly or after any combination of the following: (a) conversion to another language, code or notation; (b) reproduction in a different material form; and/or (c) decompression. To this extent, program code can be embodied as one or more types of program products, such as an application/software program, component software/a library of functions, an operating system, a basic I/O system/driver for a particular computing and/or I/O device, and the like.

Control system 12 is shown including an input module 12A, an adjustment module 12B, and an operation module 12C. In general, operation module 12C operates purification system 16 to purify medium 20. Further, input module 12A receives data from transportation system 13, evaluation system 14 and/or feedback system 18. In response to the received data, adjustment module 12B can make one or more adjustments to the purification of medium 20 that are implemented by operation module 12C when operating transportation system 13 and/or purification system 16. Each module 12A-C could be implemented using program code, hardware, or any combination thereof in a known manner.

Control system 12 can control the operation of transportation system 13. To this extent, operation module 12C can adjust the movement of medium 20 by turning on/off and/or adjusting the speed of one or more components of transportation system 13. Transportation system 13 can comprise any type of system for moving medium 20 through environment 10, e.g., through evaluation system 14, purification system 16, and feedback system 18. In one embodiment, medium 20 is moved through environment 10 using a conduit 22. Conduit 22 can comprise any type of channel for conveying medium 20, such as a duct, a tube, and the like. To this extent, when medium 20 comprises water, transportation system 13 can comprise any type of machine for generating a water flow along conduit 22. Similarly, when medium 20 comprises air, transportation system 13 can comprise any type of machine (e.g., a fan) for generating an air flow in conduit 22. Still further, when medium 20 comprises food, transportation system 13 can comprise a conveyor or the like, for moving medium 20 along conduit 22.

In any event, while moving through environment 10, medium 20 can initially pass through an evaluation system 14. Evaluation system 14 can evaluate one or more properties of medium 20 before it is purified. To this extent, evaluation system 14 is shown including a flow module 14A. Flow module 14A can evaluate a flow quantity of medium 20 that is passing through environment 10 (e.g., along conduit 22). Flow module 14A can comprise any type of flow meter appropriate for the particular type of medium 20 as are known in the art.

Additionally, evaluation system 14 can include an identification module 14B for diagnosing and/or identifying an impurity (e.g., hazardous biological agent). To this extent, identification module 14B can comprise any type of detection device for detecting the presence of one or more impurities in medium 20. For example, identification module 14B can comprise any combination of one or more ultraviolet radiation sources (e.g., ultraviolet light emitting diode, ultraviolet semiconductor laser, ultraviolet laser, ultraviolet mercury lamp, and/or the like) and/or one or more terahertz radiation sources and corresponding sensing devices. The radiation source(s) can radiate medium 20 and the sensing device(s) can identify the presence of a particular impurity based on a reflection of the radiation. Further, evaluation system 14 can include a level module 14C. Level module 14C can determine a concentration of one or more impurities in medium 20 using one or more components as are known in the art.

Environment 10 is also shown including a feedback system 18. Feedback system 18 can provide feedback data for one or more properties of medium 20 after it has passed through purification system 16. To this extent, feedback system 18 can include one or more of a flow module 18A, an identification module 18B and/or a level module 18C. These modules can be configured as described above with reference to the same modules in evaluation system 14. However, it is understood that while both evaluation system 14 and feedback system 18 are each shown including all three modules, either system 14, 18 could be implemented using only a subset of these modules and/or one or more additional modules not shown and discussed herein. Further, evaluation system 14 and feedback system 18 could be configured to monitor the same set (one or more) of properties or different properties of medium 20.

Regardless, environment system 14 and/or feedback system 18 can provide data on the one or more properties of medium 20 to input module 12A of control system 12. Adjustment module 12B can analyze the monitored property(ies) of medium 20 and make one or more adjustments to the purification operation. For example, based on the amount of medium 20, the presence of a particular impurity in medium 20, and/or a concentration of a particular impurity in medium 20, adjustment module 12B can adjust a desired wavelength, power, and/or duration of the ultraviolet radiation. These adjustments can then be implemented by operation module 12C.

As previously noted, operation module 12C operates purification system 16. In one embodiment, purification system 16 includes a power module 16A and a radiation module 16B. Power module 16A can include various components that provide power to a set (one or more) of ultraviolet radiation sources in radiation module 16B. In response, the ultraviolet radiation source(s) can generate ultraviolet radiation that is directed onto medium 20 to purify it. In this case, operation module 12C can operate various components in power module 16A to alter the operation of various components in radiation module 16B.

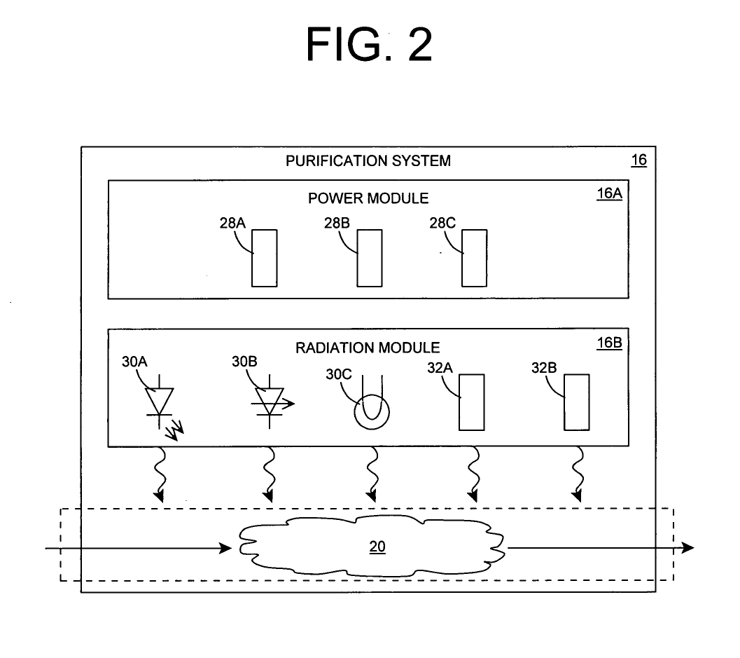

Turning to FIG. 2, a more detailed view of an illustrative purification system 16 is shown. In particular, radiation module 16B is shown including a set of ultraviolet radiation sources 30A-C. Ultraviolet radiation sources 30A-C can include one or more types of radiation sources. In one embodiment, ultraviolet radiation sources 30A-C comprise exclusively of one or more ultraviolet light emitting diodes 30A and/or one or more ultraviolet laser diodes 30B. In this manner, no mercury is required in radiation module 16B. However, in another embodiment, radiation module 16B can include one or more mercury lamps 30C, such as a low-pressure and/or medium-pressure mercury lamp, which can provide an increased power dose over a linear spectrum. In any event, the ultraviolet radiation generated by radiation module 16B can comprise one or more wavelength bands that coincide with or are close to the absorption spectra of one or more targeted biological structures (impurities) in medium 20.

Ultraviolet light emitting diode 30A can comprise, for example, a compound semiconductor ultraviolet light emitting diode or a nitride-based semiconductor ultraviolet light emitting diode. The use of ultraviolet light emitting diodes 30A and/or ultraviolet laser diodes 30B provides various benefits over the use of only a mercury lamp 30C. For example, diodes 30A and/or 30B can generate ultraviolet radiation having one or more wavelength bands that coincide with or are close to the absorption spectra of a target impurity. In one embodiment, a predominant wavelength of a radiation band generated by diodes 30A and/or 30B can be adjusted. To this extent, diodes 30A and/or 30B could generate ultraviolet radiation having a wavelength band that can be adjusted by operation module 12C (FIG. 1) based on the target impurity and its corresponding absorption spectra. For example, the wavelength band could be adjusted between approximately 200 nanometers and approximately 350 nanometers. Similarly, a power dose for the ultraviolet radiation generated by radiation module 16B, e.g., diodes 30A and/or 30B, can be adjusted based on one or more properties of medium 20. In one embodiment, the power dose can be adjusted to be between approximately 3.5 millijoules per square centimeter (mJ/cm²) and approximately 175 mJ/cm².

Power module 16A is shown including a set of power components 28A-C that can adjust the operation of radiation module 16B. In particular, power components 28A-C can be implemented as part of an electrical circuit that provides space and/or time distribution of the ultraviolet radiation generated by radiation module 16B. To this extent, the electrical circuit can control an ultraviolet power dose for the ultraviolet radiation generated by radiation module 16B. In this case, power components 28A-C can alter an amount of power applied to one or more ultraviolet radiation sources 30A-C and/or adjust an amount of distinct ultraviolet radiation sources 30A-C that generate the ultraviolet radiation. For example, power components 28A-C could include an ultraviolet photodetector, or the like, implemented as part of an electrical feedback control loop for controlling the power. Additionally, the power could be controlled by pulse switching one or more diodes 30A and/or 30B. Further, the power can be controlled based on a flow of medium 20, a type of impurity (contamination) in medium 20, a level of contamination of medium 20, and/or the like. In one embodiment, environment 10 can comprise a series of space distributed pulse-driving ultraviolet radiation sources, e.g., diodes 30A and/or 30B, with an indication and control feedback loop, which altogether provide the required purification ultraviolet dose for unstable current flows, changeable contamination, varying power supply conditions and the like.

Power module 16A can also adjust the wavelength of the ultraviolet radiation generated by radiation module 16B. For example, one or more power components 28A-C could be used to select a particular set of ultraviolet sources 30A-C that generate ultraviolet radiation having the desired wavelength. For example, the wavelength of the ultraviolet radiation generated by ultraviolet sources 30A-C can be adjusted by adjusting the band gap of the active layers of ultraviolet sources 30A-C.

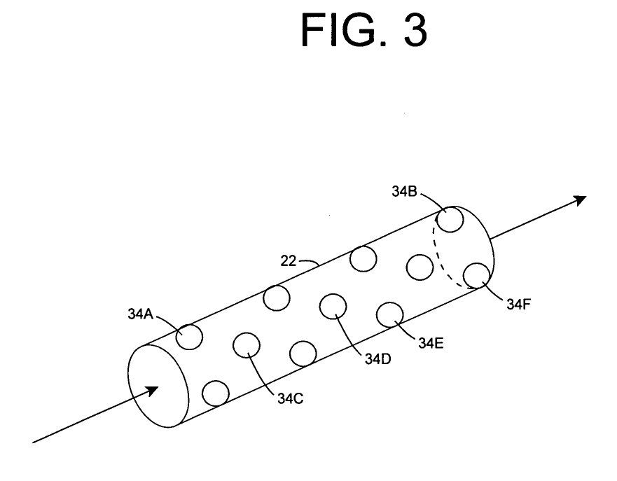

Further, since diodes 30A-B are relatively small, they can be flexibly configured. To this extent, FIG. 3 shows an illustrative conduit 22 comprising a set of ultraviolet diodes 34A-F disposed thereon. Ultraviolet diodes 34A-F could comprise one or more ultraviolet light emitting diodes 30A (FIG. 2) and/or one or more ultraviolet laser diodes 30B (FIG. 2). It is understood that the number and arrangement of ultraviolet diodes 34A-F is only illustrative, and any number and/or configuration of ultraviolet diodes 34A-F can be disposed on conduit 22. In any event, ultraviolet diodes 34A-F are configured to direct ultraviolet radiation onto the medium in conduit 22 as it passes there through.

Returning to FIG. 2, environment 10 (FIG. 1) can combine ultraviolet radiation purification with one or more purification solutions that use non-ultraviolet radiation. To this extent, radiation module 16B can comprise one or more non-ultraviolet radiation sources 32A-B. For example, radiation module 16B can comprise an X-ray radiation source 32A and/or an ionizing radiation source 32B. In this case, one or more power components 28A-C can be used to adjust the operation of radiation sources 32A-B in response to a signal or the like received from operation module 12C (FIG. 1). For example, based on the presence of a particular impurity, it may be desirable to direct X-ray and/or ionizing radiation onto medium 20 to purify it. To this extent, non-ultraviolet radiation sources 32A-B can be used to generate the appropriate non-ultraviolet radiation.

As noted previously, medium 20 can comprise air, water, food, an object, or the like. Through the use of ultraviolet light emitting diode(s) 30A and/or ultraviolet laser diode(s) 30B, radiation module 16B is made more efficient and safer than when radiation module 16B includes one or more mercury lamps 30C. To this extent, radiation module 16B can be used in field, transportation, individual, and/or portable applications. Additionally, diodes 30A-B generally provide a longer operating lifetime over that provided by mercury lamps 30C. Even further, the ultraviolet radiation parameters, such as wavelength, power, exposure time, radiation area, etc., of diodes 30A-B can be effectively controlled thereby providing an improved operating efficiency based on the particular absorption spectra of a targeted impurity (biostructure).

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

1. A system for purifying a medium, the system comprising:
a radiation module that includes a series of ultraviolet radiation sources for generating ultraviolet radiation to purify the medium, wherein the series of ultraviolet radiation sources includes:
a first ultraviolet radiation source for emitting ultraviolet radiation onto the medium the first ultraviolet radiation source including a set of ultraviolet diodes that includes at least one of an ultraviolet light emitting diode or an ultraviolet laser diode; and
a second ultraviolet radiation source for emitting ultraviolet radiation onto the medium the second ultraviolet radiation source including a mercury lamp that emits ultraviolet radiation having an increased power dose than the ultraviolet radiation emitted from the first ultraviolet radiation source.

2. The system of claim 1, wherein the medium comprises at least one of: water, air or food.

3. (canceled)

4. The system of claim 1, wherein the radiation module further comprises at least one of: an X-ray source or an ionizing radiation source.

5. The system of claim 1, wherein the ultraviolet radiation comprises a wavelength between approximately 200 nanometers and approximately 350 nanometers.

6. The system of claim 1, further comprising a power module that provides time distribution of the ultraviolet radiation.

7. The system of claim 1, further comprising a power module that controls an ultraviolet power dose to be between approximately 3.5 mJ/cm² and approximately 175 mJ/cm².

8. The system of claim 1, further comprising an evaluation system for evaluating at least one property of the medium before it is purified.

9. The system of claim 8, further comprising a control system for adjusting operation of the radiation module based on the at least one property.

10. The system of claim 9, further comprising a feedback system for providing feedback for at least one property of the medium to the control system.

11. The system of claim 1, wherein the radiation module comprises a conduit on which the set of ultraviolet diodes are disposed to direct the ultraviolet radiation on the medium in the conduit.

12-18. (canceled)

19. A system for purifying a medium, the system comprising:
a radiation module that includes a set of ultraviolet radiation sources for generating ultraviolet radiation to purify the medium, wherein the set of ultraviolet radiation sources includes:
a set of ultraviolet diodes including at least one of an ultraviolet light emitting diode or an ultraviolet laser diode for emitting ultraviolet radiation onto the medium when the medium is located at a first location; and
a mercury lamp for emitting ultraviolet radiation onto the medium when the medium is located at a second location distinct from the first location;
a control system for operating the radiation module; and
a transportation system for moving the medium through the radiation module, the transportation system including a tube on which the set of ultraviolet diodes is disposed.

20. The system of claim 19, further comprising an evaluation system for evaluating at least one property of the medium before it is purified, wherein the control system adjusts the operation of the set of ultraviolet radiation sources based on the at least one property.

21. The system of claim 19, further comprising a feedback system for providing feedback for at least one property of the medium to the control system.

22. (canceled)

23. The system of claim 19, wherein the radiation module further comprises at least one of: an X-ray source or an ionizing radiation source.

24. The system of claim 19, further comprising an evaluation system that identifies a presence of an impurity in the medium, wherein the control system adjusts operation of the set of ultraviolet diodes to generate ultraviolet radiation having at least one wavelength band that is based on an absorption spectra of the impurity.

25. The system of claim 20, wherein the at least one property comprises a flow quantity of the medium.

26. The system of claim 1, wherein the series of ultraviolet radiation sources are spatially distributed such that the first ultraviolet radiation source emits ultraviolet radiation onto the medium when the medium is located at a first location, and the second ultraviolet radiation source emits ultraviolet radiation onto the medium when the medium is located at a second location distinct from the first location.

27. The system of claim 26, wherein the set of ultraviolet diodes are disposed on a tube containing the medium and allowing the medium to pass there through.

28. A system for purifying a medium, the system comprising:
a conduit for containing the medium and allowing the medium to pass there through, the conduit including a tube and the medium comprising at least one of a liquid or a solid;
a radiation module that includes a series of ultraviolet radiation sources for generating ultraviolet radiation to purify the medium, wherein the series of ultraviolet radiation sources includes:
a first ultraviolet radiation source for emitting ultraviolet radiation onto the medium in the tube, the first ultraviolet radiation source including a plurality of ultraviolet diodes disposed on the tube; and
a second ultraviolet radiation source for emitting ultraviolet radiation onto the medium, the second ultraviolet radiation source including a mercury lamp that emits ultraviolet radiation having an increased power dose than the ultraviolet radiation emitted from the first ultraviolet radiation source;
a control system for operating the radiation module; and
an evaluation system for evaluating at least one property of the medium before it is purified, wherein the control system adjusts operation of the series of ultraviolet radiation sources based on the at least one property.

29. The system of claim 28, further comprising a transportation system for moving the medium through the conduit.

30. The system of claim 28, further comprising a feedback system for providing feedback for at least one property of the medium after it is purified to the control system, wherein the control system adjusts operation of the radiation module based on the feedback.

31. The system of claim 28, wherein the radiation module further includes at least one of:
an X-ray source or an ionizing radiation source.

32. The system of claim 28, wherein the evaluation system further identifies a presence of an impurity in the medium, and wherein the control system adjusts operation of the plurality of ultraviolet diodes to generate ultraviolet radiation having at least one wavelength band that is selected based on an absorption spectra of the impurity.
